(19) United States
(12) Patent Application Publication
(10) Pub. No.: US 2011/0251399 A1
(43) Pub. Date: Oct. 13, 2011

(54) METHOD FOR PRODUCING ALDEHYDES AND KETONES FROM PRIMARY AND SECONDARY ALCOHOLS

(75) Inventors: Uwe Dingerdissen, Seeheim (DE); Jan Pfeffer, Essen (DE); Thomas Tacke, Alzenau (DE); Thomas Haas, Muenster (DE); Harald Schmidt, Muelheim an der Ruhr (DE); Florian Klasovsky, Haltern am See (DE); Roger Sheldon, Hoog-Kappel (NL); Michiel Janssen, Den Haag (NL); Michael Volland, Duelmen (DE); Michael Rimbach, Herne (DE); Stefanie Rinker, Huenxe (DE)

(73) Assignee: EVONIK DEGUSSA GMBH, ESSEN (DE)

(21) Appl. No.: 13/140,921

(22) PCT Filed: Jan. 25, 2010
(86) PCT No.: PCT/EP10/50772

§ 371 Date: Jun. 20, 2011

(30) Foreign Application Priority Data
Feb. 6, 2009 (DE) 10 2009 000662.1

(51) Int. Cl. C07D 401/04; C07C 45/78; C07D 307/68; C07C 45/82; C07C 45/38; C07C 45/61; C07C 45/81

(52) U.S. Cl. 546/276.4; 568/354; 568/397; 568/471; 549/498

(57) ABSTRACT

The invention relates to a method for producing aldehydes and ketones from easily accessible primary and secondary alcohols by oxidation with atmospheric oxygen or pure oxygen using a catalyst system which consists of a derivative of a free nitroxyl radical.

The present invention describes a process for producing aldehydes and ketones from inexpensively available primary and secondary alcohols by oxidation with atmospheric oxygen or pure oxygen using a catalyst system consisting of a free nitroxyl radical derivative.

Stable nitroxyl radical derivatives were first described by Hoffmann (A. K. Hoffmann, A. T. Henderson, J. Am. Chem. Soc. 83 (1961) 4671) and Lebeder (O. L. Lebeder, S. N. Kazarnovskii, Zh. Obshch. Khim. 30 (1960) 1631; O. L. Lebeder, S. N. Kazarnovskii, C A 55 (1961) 1473a.). They were first used as radical scavengers. Their usefulness as catalysts for the oxidation of alcohols was only discovered recently (e.g., J. M. Bobbitt, C. L. Flores, Heterocycles 27 (1988) 509 or A. E. J. de Nooy, A. C. Besemer, H. van Bekkum, Synthesis (1996) 1153).

One disadvantage with this type of catalysis is that the oxygen required for oxidation frequently is generated from expensive sources of oxygen. The use of hypochlorite, chloroperbenzoic acid, peroxomonosulfuric acid, periodic acid or trichloroisocyanuric acid for example has been reported (e.g. L. Anelli, C. Biffi, F. Montanari, S. Quici, J. Org. Chem. 52 (1987) 2559; J. A. Cella, J. A. Kelley, E. F. Kenehan, J. Org. Chem. 40 (1975) 1850; S. D. Rychovsky, R. Vaidyanathan, J. Org. Chem. 64 (1999) 310; Bolm, Carsten; Magnus, Angelika S.; Hildebrand, Jens P. Organic Letters (2000), 2(8), 1173-1175; S. S. Kim, K. Nehru, Synletter (2002) 616; De Luca, Lidia; Giacomelli, Giampaolo; Porcheddu, Andrea. Organic Letters (2001), 3(19), 3041-3043). In addition, many of the reagents mentioned contain halogens (chlorine, bromine and iodine in particular) which, under the reaction conditions, can have a corrosive effect and often lead to undesired secondary reactions.

The oxidation of alcohols with oxygen by using nitroxyl radical derivatives is accomplished through addition of transition metals, for example cobalt, copper, tungsten, ruthenium, manganese and iron (e.g., Sheldon Roger A.; Arends, Isabel W. C. E. Journal of Molecular Catalysis A: Chemical (2006), 251(1-2), 200-214; Minisci, Francesco; Punta, Carlo; Recupero, Francesco. Journal of Molecular Catalysis A: Chemical (2006), 251(1-2), 129-149). One disadvantage with this method is the frequently difficult removal of the transition metal salts and their toxic properties.

Augustine (U.S. Pat. No. 7,030,279) describes in general the oxidation of primary or secondary alcohols with oxygen as oxidant to the corresponding aldehydes and ketones using a catalyst system consisting of a free nitroxyl radical derivative, a nitrate source, a bromine source and a carboxylic acid, which is acetic acid in all cases.

Xinquan Hu et al. describe in J. AM. CHEM. SOC. 2004, 126, 4112-4113 the alcohol oxidation using nitroxyl radical derivative and oxygen, wherein the nitrate source was replaced by a nitrite source. It is explicitly stated that the use of a bromine source is indispensable. The solvent mentioned is dichloromethane in the examples described.

The use of halogen sources is indispensable in the method of Augustine and Xinquan Hu.

The halogen-free and transition metal-free catalysis using nitroxyl radical derivatives and oxygen is described in Fried (U.S. Pat. No. 5,136,103 and U.S. Pat. No. 5,155,280), wherein nitric acid is used as a further reagent. However, the ketone yields reported are below 90% and conversions were only between 24 and 72% in the case of the aldehydes. This method is more useful for synthesizing carboxylic acids (U.S. Pat. No. 5,239,116). It is only when nitric acid is used as a stoichiometric reagent, i.e., when oxygen is omitted as an inexpensive oxidant (U.S. Pat. No. 5,155,279), conversions of 42-84% are reported at selectivities of 69-81%.

The prior art processes have the disadvantage that they are frequently unsuitable for large scale industrial production of aldehydes and ketones owing to the low yields and many process steps or the stoichiometric use of costly, corrosive and poisonous reagents. Particularly the addition of halogen sources which is needed in the nitroxyl radical-catalyzed reaction of alcohols, in conjunction with carboxylic acids, but also in organic solvents, is an extremely corrosive system which makes implementation on an industrial scale difficult. Alternatively, poisonous and difficult-to-remove transition metals are frequently used as catalyst constituent.

It is an object of the present invention to provide a process for producing aldehydes and ketones which allows the use of primary or secondary alcohols as a starting material and which is such that the addition of halogen sources, more particularly bromine sources and the use of transition metals can be dispensed with.

We have found that this object is achieved, surprisingly, by a process for producing aldehydes and ketones which allows the use of primary and secondary alcohols as a starting material and which overcomes precisely the abovementioned disadvantages of the prior art.

The present invention accordingly provides a process for producing aldehydes and ketones comprising the oxidation of primary or secondary alcohols with an oxygen-containing gas in the presence of a catalyst composition comprising at least one nitroxyl radical, one or more NO sources and at least one or more carboxylic acids or anhydrides and/or mineral acids or anhydrides, optionally in the presence of one or more solvents, characterized in that the primary and secondary alcohols used have a value of less than 2 for the decadic logarithm of the n-octanol-water partition coefficient (log P), and in that the aldehydes and ketones are preferably obtained in a yield of more than 92%, based on the alcohol used.

The process of the present invention has the advantage that the alcohol is oxidized using a mild method in the presence of nitroxyl radicals in a single process step. This mild method surprisingly makes it possible to oxidize even sterically hindered alcohols which are not available using other methods. This oxidation frequently provides almost quantitative conversions and selectivities, i.e., the high yields testify to the high efficiency of the process according to the present invention. This high efficiency is completely surprising on the basis of the prior art.

More particularly, corrosive sources of halogen are avoided as is the use of transition metals, and the substrate which constitutes the NO source is adjusted to the particular process requirements so as to avoid the formation of salt contents which are difficult and hence energy-intensive to work up. A particular advantage, however, is that the process proceeds without addition of external sources of halogen, i.e., in a halogen-free manner. Halogen sources for the purposes of the present invention means any halogen-containing compound capable of releasing halogens in elemental form or halogen-containing ions in any oxidation state. An addition of halogen-containing compounds, such as chlorites or bromine-containing compound such as N-bromosuccinimide, N-bromo-phthalimide, tetrabutylammonium bromide or inorganic salts, for example NH₄Br, alkali or alkaline earth metal bromides to carry out the oxidation can accordingly be omitted, contrary to the prior art.

It follows that the process product obtained is also very substantially halogen free in that halogen free for the purposes of the present invention means that no halogen comes from an external source of halogen.

The process step of the process of the present invention utilizes primary and secondary alcohols, for which the use of polyfunctional alcohols and poly(alcohol)s is also possible. It is a particular advantage that the process of the present invention achieves the conversion of sterically demanding alcohols into alcohols/ketones. The fundamental condition for an alcohol to be usable is that it have a value of less than 2.4 and preferably of less than 2 for the decadic logarithm of the n-octanol-water partition coefficient (log P). The n-octanol-water partition coefficient K_{ow} or P is a dimensionless partition coefficient which indicates the ratio of the concentrations of a chemical in a binary system composed of 1-octanol and water (see J. Sangster, Octanol-Water Partition Coefficients: Fundamentals and Physical Chemistry, Vol. 2 of Wiley Series in Solution Chemistry, John Wiley & Sons, Chichester, 1997).

The K_{ow}/P value only ever relates to one species of a chemical, and is represented by the following equation:

$${K}_{OW} = P = \frac{{C}_{o}^{{S}_{i}}}{{C}_{w}^{{S}_{i}}}$$

where
C_{O}^{Si}=concentration of species i of a chemical in the octanol-rich phase,
C_{w}^{Si}=concentration of species i of a chemical in the water-rich phase
   P is generally reported in the form of the decadic logarithm as log P (or else log P_{ow} or more rarely log pOW):

The K_{ow} value is a model measure of the ratio between the lipophilicity (fat solubility) and hydrophilicity (water solubility) of a substance. The expectation is that the partition coefficient of a substance in the octanol-water system will make it possible to estimate the partition coefficients of this substance in other systems involving an aqueous phase. K_{ow} is greater than one when a substance is more soluble in fatlike solvents such as n-octanol and less than one when it is more soluble in water. Accordingly, Log P is positive for lipophilic substances and negative for hydrophilic substances. Since the KOW cannot be measured for all chemicals, there are various predictive models, for example via quantitative structure-activity relationships (QSARs) or via linear free energy relationships (LFERs), described for example in Eugene Kellogg G, Abraham D J: Hydrophobicity: is Log P(o/w) more than the sum of its parts? Eur J Med. Chem. 2000 July-August; 35(7-8):651-61 or Gudrun Wienke, Measurement and Predictive Computation of n-Octanol/Water Partition Coefficients [Messung and Vorausberechnung von n-Octanol/Wasser-Verteilungskoeffizienten], PhD thesis, Oldenburg University, 1-172, 1993.

In the context of the present invention, log P is determined according to the method of Advanced Chemistry Development Inc., Toronto using the ACD/Log P DB program module.

Examples of appropriate alcohols include lower aliphatic alcohols such as ethanol, ethylene glycol and glycerol for example, but also higher molecular weight systems such as sugar alcohols, for example isosorbitol, isomannitol and derivatives thereof, or polyols, for example polyethylene glycols.

The oxidation of the process according to the invention is performed using a catalyst composition in the absence of transition metals.

Nitroxyl radicals are an essential constituent part of the catalyst composition used in the process of the present invention. For the purposes of this invention, nitroxyl radicals are compounds which contain the moiety

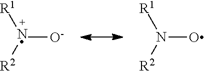

and which at room temperature are stable in the presence of oxygen for at least one week. These nitroxyl radicals have no hydrogen atoms on the α-C-atom next to the nitrogen atom.

The nitroxyl radicals used in the catalyst composition of the process according to the present invention preferably comprise compounds having the structure (I) and/or saltlike compounds having the structure (II):

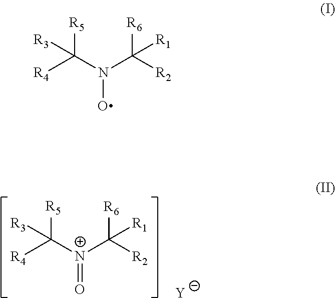

where
R¹, R², R³, R⁴, R⁵ and R⁶=(C₁-C₁₀)-alkyl, (C₁-C₁₀)-alkenyl, (C₁-C₁₀)-alkoxy, (C₆-C₁₈)-aryl, (C₇-C₁₉)-aralkyl, (C₆-C₁₈)-aryl-(C₁-C₈)-alkyl or (C₃-C₁₈-heteroaryl, wherein the substituents of the type R¹, R², R³, R⁴, R⁵ and R⁶ are the same or different and the substituents of the type R⁵ and R⁶ can also combine to form a (C₁-C₄)-alkylene bridge, which can be saturated or unsaturated, unsubstituted or substituted, particularly with one or more substituents selected from R¹, C₁-C₈-amido, halogen, oxy, hydroxyl, amino, alkylamino, dialkylamino, arylamino, diarylamino, alkylcarbonyloxy, arylcarbonyloxy, alkylcarbonylamino and arylcarbonyl-amino. In structure (II) Y⁻ is any desired halogen-free anion.

The process of the present invention can utilize not only one nitroxyl radical but also a mixture of various nitroxyl radicals.

The nitroxyl radicals used in the process of the present invention preferably include 2,2,6,6-tetra-methylpiperidine-1-oxyl (TEMPO) and/or the 2,2,6,6-tetramethylpiperidine-1-oxyl derivatives substituted at position 4 of the heterocycle, wherein the derivatives display one or more substituents selected from R¹, C₁-C₈-amido, halogen, oxy, hydroxyl, amino, alkylamino, dialkylamino, arylamino, diarylamino, alkylcarbonyloxy, arylcarbonyloxy, alkylcarbonylamino and arylcarbonyl-amino groups, where R¹ is a (C₁-C₁₀-alkyl, (C₁-C₁₀)-alkenyl, (C₁-C₁₀)-alkoxy, (C₆-C₁₀)-aryl, (C₇-C₁₉)-aralkyl, (C₆-C₁₈)-aryl-(C₁-C₈)-alkyl or (C₃-C₁₈)-heteroaryl group. Examples of appropriate compounds include 4-methoxy-2,2,6,6-tetramethylpiperidine-1-oxyl (4-MeO-TEMPO), 4-oxo-2,2,6,6-tetramethylpiperidine-1-oxyl (4-oxo-TEMPO), 4-hydroxy-2,2,6,6-tetramethylpiperidine-1-oxyl (4-hydroxy-TEMPO), 4-benzoyloxy-2,2,6,6-tetramethyl-piperidine-1-oxyl (BnO-TEMPO), 4-acetamido-2,2,6,6-tetramethylpiperidine-1-oxyl, 4-acetamino-2,2,6,6-tetramethylpiperidine-1-oxyl (AA-TEMPO), 4-amino-2,2,6,6-tetramethylpiperidine-1-oxyl, N,N-dimethyl-amino-2,2,6,6-tetramethylpiperidine-1-oxyl (NNDMA-TEMPO), 3,6-dihydro-2,2,6,6-tetramethyl-1(2H)-pyridinyloxyl (DH-TEMPO) or bis(2,2,6,6-tetramethyl-piperidine-1-oxyl-4-yl) sebacate, and may include one or more substituents selected from R¹, C₁-C₈-amido, halogen, oxy, hydroxyl, amino, alkylamino, dialkylamino, arylamino, diarylamino, alkylcarbonyloxy, arylcarbonyloxy, alkylcarbonylamino and arylcarbonyl-amino.

The tetramethylpiperidine-N-oxyl structural fragment conforming to structures (I) or (II) can also be a constituent part of a larger macromolecule, of an oligomer or else of a polymeric structure. An example of such a nitroxyl radical is shown by structure (III):

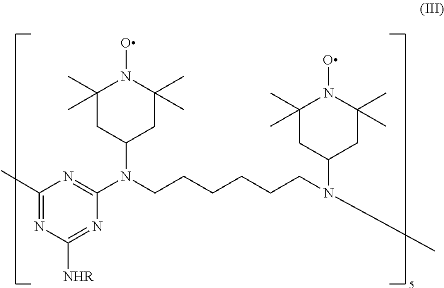

The “heterogenized” form of the nitroxyl radicals can also be used in the process of the present invention. This means that the nitroxyl radicals are supported on, for example, aluminum oxide, silicon dioxide, titanium dioxide or zirconium dioxide. Polymers, composites or carbon materials can also be used as a support material for the nitroxyl radical.

The process of the present invention preferably utilizes the abovementioned compounds AA-TEMPO, 4-hydroxy-TEMPO, TEMPO and 4-oxo-TEMPO as nitroxyl radicals. Particular preference is given to using AA-TEMPO, 4-hydroxy-TEMPO and TEMPO, more particularly AA-TEMPO.

The proportion of nitroxyl radical in the process of the present invention is preferably in the range from 0.001 to 10 mol %, more preferably in the range from 0.01 to 5 mol % and even more preferably in the range from 0.1 to 2 mol %, based on the amount of alcohol used.

The catalyst composition used in the process of the present invention further comprises at least one NO source. In the process of the present invention, the NO source used may comprise ammonium nitrate or nitrite, alkali or alkaline earth metal nitrates or nitrites, for example magnesium nitrate, or sodium nitrite. In addition to the nitrates or nitrites or as replacement for the nitrates or nitrites it is possible to use nitrous gases such as N₂O, NO, N₂O₃, NO₂, N₂O₄, and N₂O₅. Useful NO sources further include mixtures of various of the NO sources mentioned above. The proportion of NO source(s) used in the process of the present invention is in the range from 0.001 to 10 mol %, preferably in the range from 0.01 to 5 mol % and more preferably in the range from 0.1 to 2 mol %, based on the amount of alcohol used.

The catalyst composition used in the process of the present invention further comprises at least one or more carboxylic acids and/or anhydrides and/or mineral acids or anhydrides. The process of the present invention preferably utilizes acetic acid or acetic anhydride, propionic acid or some other carboxylic acid or anhydride that dissolves in the reaction mixture as carboxylic acid or carboxylic anhydride. The process of the present invention preferably utilizes acetic acid. Mixtures of various suitable carboxylic acids or solutions of carboxylic acids in a suitable solvent can also be used. The amount of carboxylic acid used is preferably in the range from 0.1 to 200 mol % and more preferably in the range from 10 to 50 mol %, based on the amount of alcohol used.

For the purposes of the present invention, the term “mineral acids” is used as a collective name for all inorganic acids. Suitable mineral acids include for example H₂CO₃, H₃PO₄, HNO₃, HNO₂, H₂SO₄, H₂SO₃, H₃BO₃, their anhydrides or mixtures thereof.

The oxidizing agent used in the process of the present invention is an oxygen-containing gas. Pure oxygen can be used as oxygen-containing gas, but it is also possible to use mixtures of oxygen with an inert gas or air or with a gas involved in the reaction. Suitable inert gases include for example nitrogen, carbon dioxide, helium or argon. Examples of gases involved in the reaction include nitrogen oxides as mentioned above in the description of the NO sources. The oxygen partial pressure is preferably in the range from 0.1 to 100 bar and more preferably in the range from 0.2 to 50 bar.

The process of the present invention can be carried out in a solvent or without use of a solvent:

In one particular embodiment of the process according to the present invention, the process step is carried out in the presence of a solvent. Preference is here given to using polar solvents, more particularly polar organic solvents. Preference for use as solvents is given to acetonitrile, tetrahydrofuran, ethyl acetate, acetone, diethyl ether, methyl tert-butyl ether, tertiary alcohols such as tert-amyl alcohol, tert-butyl alcohol, esters of carbonic acid such as dimethyl carbonate, diethyl carbonate, hydrocarbons, or a mixture thereof. The amount of solvent used is preferably in the range from 0.1% to 70% by volume, more preferably in the range from 0.5% to 60% by volume and most preferably in the range from 1% to 50% by volume, based on the amount of alcohol used.

In a further preferred embodiment of the process according to the present invention, no additional solvent is used in the process step. In this case, the carboxylic acid or the mineral acid serves not just as a component of the catalyst composition but also as a solvent to keep the reaction mixture homogeneous. This has the advantage that the use of solvents that are flammable and possibly hazardous to health can be dispensed with, and/or special removal of the solvent can be omitted.

In what follows, the practice of the process according to the present invention is elucidated. The oxidation of the process according to the present invention is preferably carried out at a temperature of 0 to 100° C. or at the boiling point of the solvent.

Total pressure in the oxidation in the process of the present invention is preferably in the range from 1 to 300 bar and more preferably in the range from 1 to 50 bar.

The process can be run as a batch process, as a semi-batch process or as a continuous process. Furthermore, the process of the present invention is not limited to a certain reactor type; on the contrary, the process step can be carried out in a stirred tank, a tube reactor, a tank cascade, a micro reactor or a combination thereof.

In one embodiment of the process according to the present invention, initially the alcohol is dissolved or suspended in a suitable solvent and then the catalyst composition is added individually or as a mixture to this solution or suspension. Pressure and temperature are then adjusted. However, it is also possible to charge the catalyst composition initially and to add the solution or suspension of the alcohol to the catalyst composition. When the process of the present invention is run as a continuous process, it is preferable to feed the alcohol together with the reaction gases in the embodiment of a trickle bed.

It is particularly advantageous in the process of the present invention for the level of water in the reaction composition to be very low, since high proportions of water can reduce the yields. In addition, the reaction can be speeded by removing the water of reaction from the reaction environment.

There are several options to minimize the water content. Water-absorbing agents, preferably sodium sulfate, calcium oxide and molecular sieves, for example zeolites, can be added to the reaction mixture. Concentrated strongly hydrating acids or salts can also be used. For example, the abovementioned mineral acids and/or anhydrides can be used as water-binding acids. It is further possible to use very strongly water-complexing solvents, for example glacial acetic acid.

It is additionally also possible to use solvents capable of binding water chemically. Preferably, the anhydrides of the carboxylic acids used in the process of the present invention are concerned here. Thus the carboxylic anhydrides act not only as solvents for the reaction but at the same time also as a water-binding agent. This makes for an efficient reaction management which at the same time leads to high yields, an effect not known in that form from the prior art.

Alternatively, the water of reaction can also be withdrawn from the reaction mixture by distillative removal or by extractive removal from the reaction environment.

The reaction mixture is generally worked up in a way which depends on the polarity of the target molecules and the solubility of the nitroxyl radicals. A procedure for readily water-soluble aldehydes and ketones comprises:
a) removing the solvents and the carboxylic acid by distillation or extraction,
b) extracting and optionally recycling the nitroxyl radicals,
c) removing any salts from the NO sources via ion exchangers, electrodialysis or ultrafiltration,
d) and/or purifying the product by crystallization, distillation, extraction and/or chromatographic separation.

The process steps mentioned can be carried out alone or in any desired combination with one another.

The aldehydes and ketones obtained via the process according to the present invention are very useful as intermediates for further reaction. For instance, they can be converted, by reductive amination with ammonia, hydroxylamine or hydrazine, into the corresponding amines which are very useful as raw materials for the production of plastics.

Even without further exposition it is believed that a person skilled in the art will be able to make the widest use of the above description. Therefore, the preferred embodiments and examples are merely to be understood as a descriptive disclosure which is not in any way intended to be limiting.

The present invention will now be more particularly described with reference to examples. Alternative embodiments of the present invention are obtainable analogously.

### EXAMPLES

### a) Sterically Demanding Alcohols in Glacial Acetic Acid:

1. Oxidation of Isosorbitol with AA-Tempo/O₂/Nitrate-/Nitrite, Bromine Free

### Material:

| | | | | |
|---|---|---|---|---|
| isosorbitol (1,4:3,6-dianhydro- | 100 | mmol | → | 73.07 g/L |
| D-sorbitol) | | | | |
| AA-TEMPO | 5 | mol % | → | 5.35 g/L |
| NaNO₂ | 2 | mol % | → | 2.55 g/L |
| Mg(NO₃)₂ × 6H₂O | 2 | mol % | → | 0.70 g/L |
| acetic acid (100%) | 200 | mL | | |
| decane (internal standard for | 7 | g | → | 35 g/L |
| analysis) | | | | |

| | |
|---|---|
| logP isosorbitol | −1.67 |

### Reaction Conditions:

reaction temperature: T=50° C.
reaction time: t=4 h
oxygen: 10 bar
reactor volume: 450 mL

### Procedure:

The reactant, the catalyst, the salts and the internal standard are initially charged, dissolved in the acetic acid (addition of acetic acid until the volume of 200 ml is reached) and transferred into the high pressure reactor. The reactor is sealed and twice inertized with nitrogen. The solution is then heated to a temperature of 50° C. under agitation. The reaction is started by injecting 10 bar of oxygen into the autoclave. The pressure in the reactor is kept at a constant 10 bar by replenishing consumed oxygen via an opened oxygen supply up to a pressure of 10 bar. After four hours, the reaction is discontinued by inertization with nitrogen and cooling down of the system.

### Result:

A conversion of above 97% is observed at a 100% selectivity of the diketone (2,6-dioxabicyclo-(3.3.0)-octane-4,8-dione).

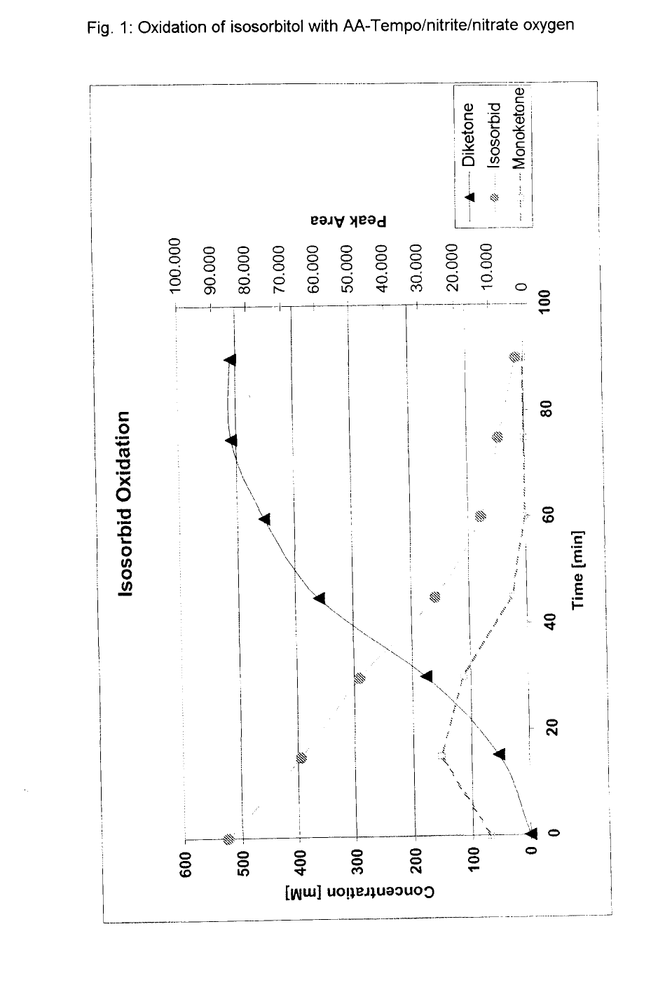

FIG. 1 shows the development of the isosorbitol, diketone and monoketone concentrations as a function of time during the performance of the process of the present invention. The isosorbitol and diketone concentrations were evaluated via calibration curves and the internal standard. The monoketone concentrations are evaluated via the peak areas.

2. Oxidation of Isomannitol with AA-Tempo/O₂/Nitrite-/Nitrate, Bromine Free:

### Material:

| | | | | |
|---|---|---|---|---|
| isomannitol (1,4:3,6-dianhydro- | 25 | mmol | → | 73.07 g/L |
| D-mannitol) | | | | |
| AA-TEMPO | 5 | mol % | → | 5.35 g/L |
| NaNO₂ | 2 | mol % | → | 2.55 g/L |
| Mg(NO₃)₂ × 6H₂O | 2 | mol % | → | 0.70 g/L |
| acetic acid (100%) | 50 | mL | | |
| decane (internal standard for | 1.75 | g | → | 35 g/L |
| analysis) | | | | |

| | |
|---|---|
| logP isomannitol | −1.67 |

### Reaction Conditions:

reaction temperature: T=50° C.
reaction time: t=1.5 h
oxygen: 10 mL/min

### Procedure:

The reactant, the catalyst, the salts and the internal standard are initially charged, dissolved in the acetic acid (addition of acetic acid until the volume of 50 ml is reached) and transferred into a four neck flask equipped with reflux condenser, thermometer, gas inlet frit and septum for sample taking. The solution is then heated to a temperature of 50° C. under agitation. The reaction is started by introducing oxygen into the reaction solution via the gas inlet frit at a flow rate of 10 ml/min. The reaction is run for a period of 1.5 h during which a sample is taken every 15 min to record the kinetics.

### Result:

A conversion of 100% is observed at a 100% selectivity of the diketone (2,6-dioxabicyclo-(3.3.0)-octane-4,8-dione).

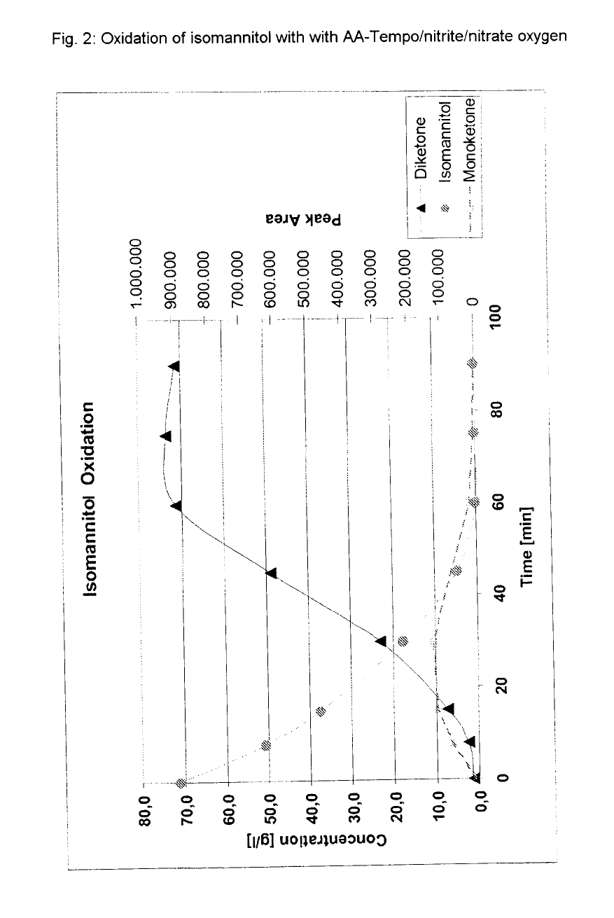

FIG. 2 shows the development of the isomannitol, diketone and monoketone concentrations as a function of time during the performance of the process of the present invention. The isomannitol and diketone concentrations were evaluated via calibration curves and the internal standard. The monoketone concentrations are evaluated via the peak areas.

3. Oxidation of Isosorbitol with Various N-Oxy Radicals/O₂/Nitrite/Nitrate Bromine Free

### Material:

| | | | | |
|---|---|---|---|---|
| isosorbitol (1,4:3,6-dianhydro- | 10 | mmol | → | 73.07 g/L |
| D-sorbitol) | | | | |
| N-oxy radical | 5 | mol % | | |
| NaNO₂ | 2 | mol % | → | 2.55 g/L |
| Mg(NO₃)₂ × 6H₂O | 2 | mol % | → | 0.70 g/L |
| acetic acid (100%) | 20 | mL | | |
| hexadecane (internal standard | 0.05 | g | → | 2.5 g/L |
| for analysis) | | | | |

| | |
|---|---|
| logP isosorbitol | −1.67 |

### Reaction Conditions:

reaction temperature: T=50° C.
reaction time: t=1.5 h-4.5 h
oxygen: 10 mL/min

### Procedure:

The reactant, the catalyst and the salts are initially charged to a three neck flask equipped with a reflux condenser and a gas inlet frit, admixed with 20 ml of acetic acid and dissolved under agitation. The solution is then heated to a temperature of 50° C. under agitation. The reaction is started by introducing oxygen into the reaction solution via the gas inlet frit at a flow rate of 20 ml/min. The reactions are run for a period of 1.5 h-4.5 h during which a sample is taken every 30 min to record the kinetics.

### Result:

Various N-oxy radicals are used for the reaction in accordance with the protocol described above. The reaction is run for a period of 1.5 h-4.5 h depending on the N-oxy radical used. The N-oxy radical AA-Tempo gives a conversion of 100% and a 100% selectivity to the diketone.

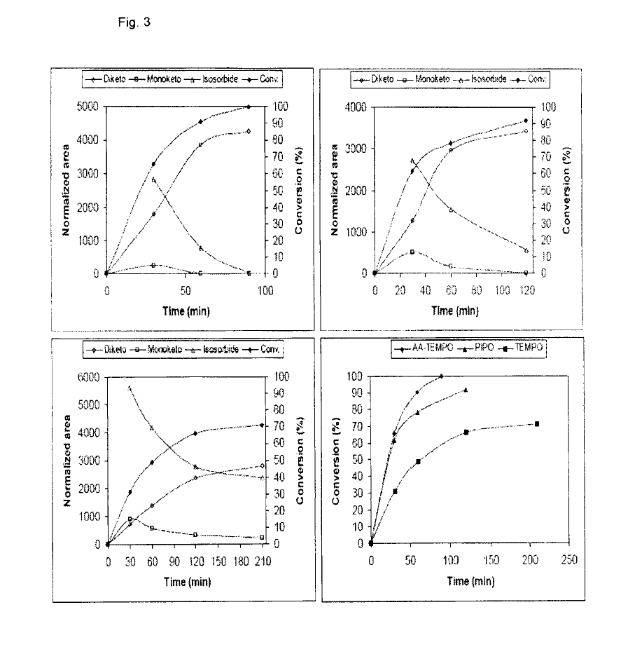

FIG. 3 shows the results of comparative tests in respect of the reactions. Plots are shown of the oxidation of isosorbitol with AA-Tempo (at top left), PIPO (at top right), Tempo (at bottom left) and also a comparison of the conversions of all three N-oxy radicals as a function of time. The graphs show the normalized areas from GC analysis and the conversion of the isosorbitol in % evaluated using hexadecane as internal standard.

4. Oxidation of Isosorbitol with AA-Tempo/O₂/Nitrite, Bromine Free

### Material:

| | | | | |
|---|---|---|---|---|
| isosorbitol (1,4:3,6-dianhydro- | 100 | mmol | → | 73.07 g/L |
| D-sorbitol) | | | | |
| AA-TEMPO | 5 | mol % | → | 5.35 g/L |
| NaNO₂ | 2.5 | mol % | → | 3.19 g/L |
| acetic acid (100%) | 200 | mL | | |
| decane (internal standard for | 7 | g | → | 35 g/L |
| analysis) | | | | |

| | |
|---|---|
| logP isosorbitol | −1.67 |

### Reaction Conditions:

reaction temperature: T=50° C.
reaction time: t=4 h
oxygen: 10 bar
reactor volume: 450 mL

### Procedure:

The reactant, the catalyst, the salts and the internal standard are initially charged, dissolved in the acetic acid (addition of acetic acid until the volume of 200 ml is reached) and transferred into the high pressure reactor. The reactor is sealed and twice inertized with nitrogen. The solution is then heated to a temperature of 50° C. under agitation. The reaction is started by injecting 10 bar of oxygen into the autoclave. The pressure in the reactor is kept at a constant 10 bar by replenishing consumed oxygen via an opened oxygen supply up to a pressure of 10 bar. After four hours, the reaction is discontinued by inertization with nitrogen and cooling down of the system.

### Result:

A conversion of above 97% is observed coupled with 100% selectivity for the diketone (2,6-dioxabicyclo-(3.3.0)-octane-4,8-dione).

5. Oxidation of Isosorbitol with Tempo/NaOCl/Bromide (Comparative Experiment by Chlorate Method)

### Material:

| | | |
|---|---|---|
| isosorbitol (1,4:3,6-dianhydro-D- | → | 20 g/L |
| sorbitol) | | |
| Tempo | → | 1.71 g/L |
| NaBr | → | 11.30 g/L |
| NaOCl | → | 81.5 g/L |
| water | → | 100 ml |

| | |
|---|---|
| logP isosorbitol | −1.67 |

### Reaction Conditions:

reaction temperature: T=2-5° C.
reaction time: t=2 h

### Procedure:

2 g of isosorbitol, 1127 mg of sodium bromide and 171 mg of TEMPO are suspended in 100 ml of water and cooled to 0° C. in an ice-water bath. The continuously measured pH of this mixture is adjusted to exactly pH 10 by adding 0.5N aqueous sodium hydroxide solution. The run is carried out at a constant pH of 10. It is achieved through continuous titration of the resulting acids with 0.5N in aqueous sodium hydroxide solution, the pH being kept constant using an automatic titration system TITRINO, from Metrohm, Herisau (CH).

### Result:

The reaction leads to small amounts of monoketone and diketone cannot be detected.

6. Oxidation of Isosorbitol with AA-TEMPO/O₂/Nitrite-/HNO₃ (Comparative Experiment)

### Material:

| | | |
|---|---|---|
| isosorbitol (1,4:3,6-dianhydro-D- | 10 | mmol |
| sorbitol) | | |
| AA-TEMPO | 5 | mol % |
| NaNO₂ | 2 | mol % |
| HNO₃ | 10 | mol % |
| water | 20 | mL |
| hexadecane (internal standard for | 0.05 | g |
| analysis) | | |

### Reaction Conditions:

reaction temperature: T=50° C.
reaction time: t=4 h
oxygen: 20 mL/min

### Procedure:

The reactant, the catalyst, the salts and the nitric acid are initially charged to a three neck flask equipped with a reflux condenser and a gas inlet frit and filled with 10 ml of water, are made up to 20 ml with water and are dissolved under agitation. The solution is then heated to a temperature of 50° C. under agitation. The reaction is started by introducing oxygen into the reaction solution via the gas inlet frit at a flow rate of 20 mL/min. The reactions are run for a period of 4 h.

### Result:

The oxidizing system used did not give any observable conversion into either monoketone or diketone.

### b) Sterically Undemanding Alcohols in Glacial Acetic Acid

1. Oxidation of 2-Propanol with AA-Tempo/O₂/Nitric Acid, Bromine Free

### Material:

| | | | | |
|---|---|---|---|---|
| 2-propanol | 200 | mmol | → | 119.34 g/L |
| AA-TEMPO | 3.5 | mol % | → | 14.94 g/L |
| nitric acid (>99.8%) | 3.5 | mol % | → | 4.53 g/L |
| acetic acid (100%) | 100 | mL | | |

| | |
|---|---|
| logP 2-propanol | 0.38 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 15% is observed coupled with a 70% selectivity for the ketone (acetone). By-produced isopropyl acetate can be detected in a small amount.

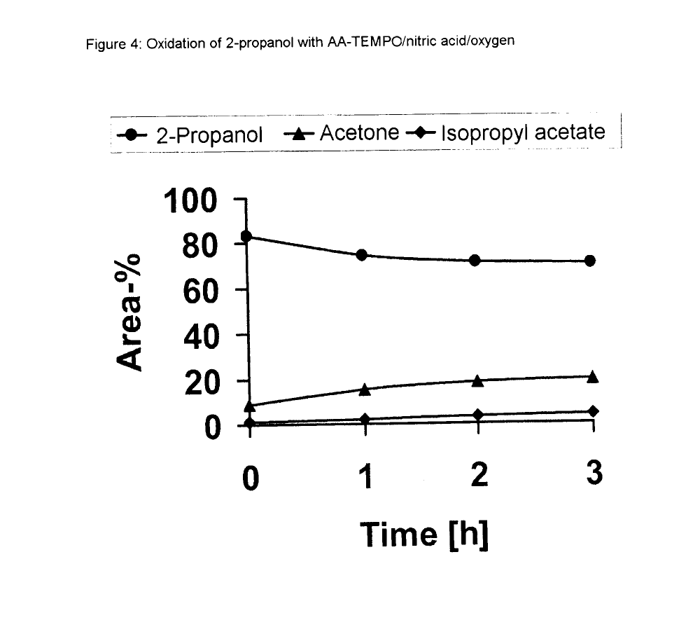

FIG. 4 shows the development of the 2-propanol, acetone and isopropyl acetate concentrations as a function of time in the performance of the process according to the present invention. The concentrations are evaluated via the peak areas.

2. Oxidation of 1-Propanol with AA-Tempo/O₂/Nitric Acid, Bromine Free

### Material:

| | | | | |
|---|---|---|---|---|
| 1-propanol | 200 | mmol | → | 119.34 g/L |
| AA-TEMPO | 3.5 | mol % | → | 14.94 g/L |
| nitric acid (>99.8%) | 3.5 | mol % | → | 4.53 g/L |
| acetic acid (100%) | 100 | mL | | |

| | |
|---|---|
| logP 1-propanol | 0.55 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 27% is observed coupled with a 20% selectivity for the aldehydes (propanal). By-produced propyl acetate can be detected in a small amount.

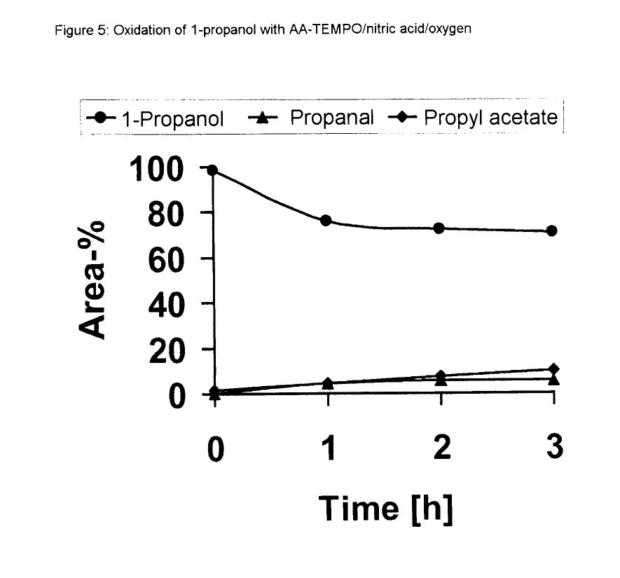

FIG. 5 shows the development of the 1-propanol, acetone and isopropyl acetate concentrations as a function of time in the performance of the process according to the present invention. The concentrations are evaluated via the peak areas.

3. Oxidation of Cyclohexanol with AA-Tempo/O₂/Nitric Acid, Bromine Free

### Material:

| | | | | |
|---|---|---|---|---|
| cyclohexanol | 200 | mmol | → | 200.32 g/L |
| AA-TEMPO | 3.5 | mol % | → | 14.94 g/L |
| nitric acid (>99.8%) | 3.5 | mol % | → | 4.53 g/L |
| acetic acid (100%) | 100 | mL | | |

| | |
|---|---|
| logP cyclohexanol | 1.27 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 14% is observed coupled with a 81% selectivity for the ketone (cyclohexanone). By-produced cyclohexyl acetate can be detected in a small amount.

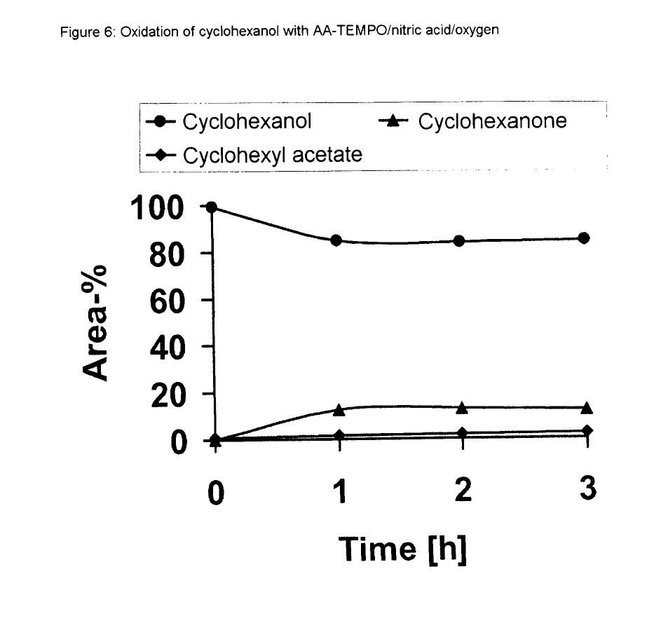

FIG. 6 shows the development of the cyclohexanol, cyclohexanone and cyclohexyl acetate concentrations as a function of time in the performance of the process according to the present invention. The concentrations are evaluated via the peak areas.

4. Oxidation of Furfuryl Alcohol with AA-Tempo/O₂/Nitric Acid, Bromine Free

### Material:

| | | | | |
|---|---|---|---|---|
| furfuryl alcohol | 200 | mmol | → | 197.8 g/L |
| AA-TEMPO | 3.5 | mol % | → | 14.94 g/L |
| nitric acid (>99.8%) | 3.5 | mol % | → | 4.53 g/L |
| acetic acid (100%) | 100 | mL | | |

| | |
|---|---|
| logP furfuryl alcohol | 0.08 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 96% is observed coupled with a 93% selectivity for the aldehydes (furfural). By-produced furfuryl acetate can be detected in a small amount.

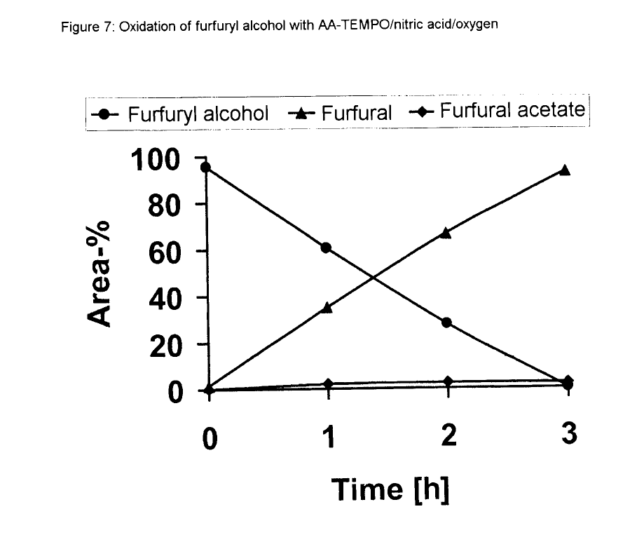

FIG. 7 shows the development of the furfuryl alcohol, furfural and furfuryl acetate concentrations as a function of time in the performance of the process according to the present invention. The concentrations are evaluated via the peak areas.

5. Oxidation of 1,3-Dihydroxycyclohexane with AA-Tempo/O₂/Nitric Acid, Bromine Free

### Material:

| | | | | |
|---|---|---|---|---|
| 1,3-dihydroxycyclohexane | 86 | mmol | → | 100 g/L |
| AA-TEMPO | 3.5 | mol % | → | 6.49 g/L |
| nitric acid (>99.8%) | 3.5 | mol % | → | 1.51 g/L |
| acetic acid (100%) | 100 | mL | | |

| | |
|---|---|
| logP 1,3-dihydroxycyclohexane | 0.01 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 11% is observed coupled with a 72% selectivity for the monooxygenated ketone (3-hydroxycyclohexanol). By-produced 2-cyclohexenone can be detected in a small amount.

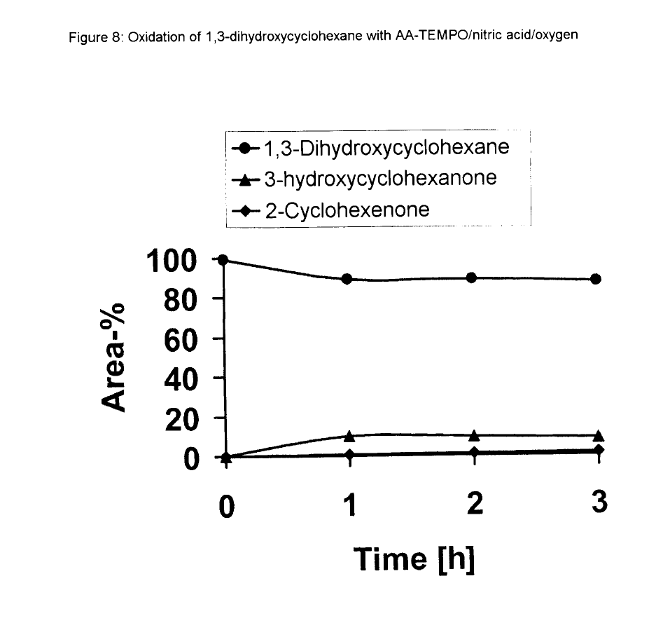

FIG. 8 shows the development of the 1,3-dihydroxycyclohexane, 3-hydroxycyclohexanone and 2-cyclohexenone concentrations as a function of time in the performance of the process according to the present invention. The concentrations are evaluated via the peak areas.

6. Oxidation of Nicotinyl Alcohol with AA-Tempo/O₂/Nitric Acid, Bromine Free

### Material:

| | | | | |
|---|---|---|---|---|
| nicotinyl alcohol | 200 | mmol | → | 218.26 g/L |
| AA-TEMPO | 3.5 | mol % | → | 14.94 g/L |
| nitric acid (>99.8%) | 3.5 | mol % | → | 4.53 g/L |
| acetic acid (100%) | 100 | mL | | |

| | |
|---|---|
| logP nicotinyl alcohol | 0.12 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 17% is observed coupled with a 79% selectivity for the aldehydes (nicotinaldehyde). By-produced nicotinyl acetate can be detected in a small amount.

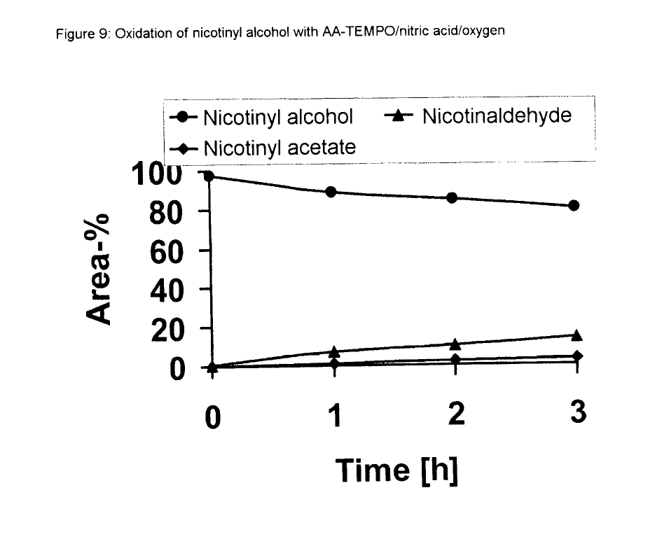

FIG. 9 shows the development of the nicotinyl alcohol, nicotinyl aldehyde and nicotinyl acetate concentrations as a function of time in the performance of the process according to the present invention. The concentrations are evaluated via the peak areas.

7. Oxidation of Borneol with AA-Tempo/O₂/Nitric Acid, Bromine Free

### Material:

| | | | | |
|---|---|---|---|---|
| borneol | 200 | mmol | → | 308.50 g/L |
| AA-TEMPO | 3.5 | mol % | → | 14.94 g/L |
| nitric acid (>99.8%) | 3.5 | mol % | → | 4.53 g/L |
| acetic acid (100%) | 100 | mL | | |

| | |
|---|---|
| logP borneol | 1.63 |

### Reaction Conditions:

reaction temperature: T=50° C.
reaction time: t=3 h
oxygen: 1 bar
reactor volume: 250 mL

### Procedure:

The reactant is initially charged, dissolved in the acetic acid (acetic acid being added until the volume of 100 ml is reached) and transferred into the reactor. The solution is then heated to a temperature of 50° C. under agitation and oxygen injection. The reaction is started by adding the acid and the catalyst to the reaction mixture. The pressure in the reactor is maintained at a constant 1 bar by replenishing consumed oxygen via an opened oxygen supply up to a pressure of 1 bar. After three hours the reaction is discontinued by interrupting the oxygen feed and cooling down of the system.

### Result:

A conversion of above 22% is observed coupled with a 82.9% selectivity for the ketone (menthol). By-produced bornyl acetate can be detected in a small amount.

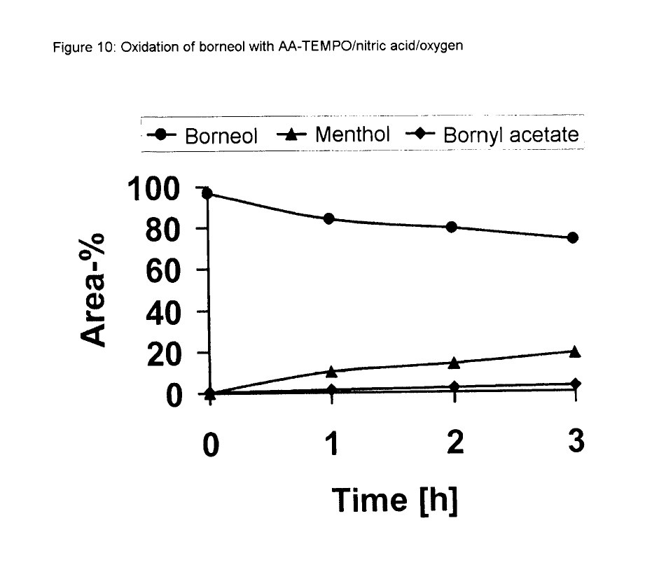

FIG. 10 shows the development of the borneol, menthol and bornyl acetate concentrations as a function of time in the performance of the process according to the present invention. The concentrations are evaluated via the peak areas.

1. A process for producing aldehydes and ketones comprising the oxidation of primary or secondary alcohols with an oxygen-containing gas in the presence of a catalyst composition comprising at least one nitroxyl radical, one or more NO sources and at least one or more carboxylic acids or anhydrides and/or mineral acids or anhydrides, optionally in the presence of one or more solvents, wherein the primary and secondary alcohols have a value of less than 2 for the decadic logarithm of the n-octanol-water partition coefficient (log P).

2. The process according to claim 1, wherein the aldehydes and ketones are obtained in a yield of more than 92%, based on the alcohol.

3. The process according to claim 1, wherein the one or more nitroxyl radicals comprise 2,2,6,6-tetramethylpiperidine-1-oxyl (TEMPO) and/or the 2,2,6,6-tetramethylpiperidine-1-oxyl derivatives substituted at position 4 of the heterocycle, wherein the derivatives display one or more substituents selected from the group consisting of R¹, C₁-C₈-amido, halogen, oxy, hydroxyl, amino, alkylamino, dialkylamino, arylamino, diarylamino, alkylcarbonyloxy, arylcarbonyloxy, alkylcarbonylamino and arylcarbonylamino groups, where R¹ is a (C₁-C₁₀)-alkyl, (C₁-C₁₀)-alkenyl, (C₁-C₁₀)-alkoxy, (C₆-C₁₈)-aryl, (C₇-C₁₉)-aralkyl, (C₆-C₁₈)-aryl-(C₁-C₈)-alkyl and (C₃-C₁₈)-heteroaryl group.

4. The process according to claim 1, wherein the proportion of nitroxyl radical is in the range from 0.001 to 20 mol %, based on the amount of alcohol.

5. The process according to claim 1, wherein at least one carboxylic acid is present as acetic acid or at least one anhydride is present as acetic anhydride.

6. The process according to claim 1, wherein the proportion of carboxylic acid and/or anhydride is in the range from 0.1 to 200 mol %, based on the amount of alcohol.

7. The process according to claim 1, wherein at least one mineral acid is present and is H₂CO₃, H₃PO₄, H₂SO₄, H₂SO₃, H₃BO₃ or their anhydrides or mixtures thereof.

8. The process according to claim 1, wherein one or more NO sources is present and comprises ammonium nitrate or nitrite, alkali or alkaline earth metal nitrates or nitrites, nitrous gases or mixtures thereof.

9. The process according to claim 1, wherein one or more NO sources is present and the proportion of NO source(s) is in the range from 0.001 to 10 mol %, based on the amount of alcohol.

10. The process according to claim 1, wherein no additional solvent is present.

11. The process according to claim 1, wherein the water of reaction is removed from the reaction mixture.

12. The process according to claim 11, wherein the water of reaction is removed by addition of water-absorbing agents to the reaction mixture and/or by distillative or extractive removal from the reaction mixture during the reaction.

13. The process according to claim 11, wherein the solvent is a water-complexing solvent or a solvent that chemically binds water.

14. The process according to claim 1, further comprising removing the carboxylic acid and, if present, the solvent by distillation or extraction, extracting and optionally recycling the nitroxyl radicals removing the salts from the NO sources and/or purifying the aldehyde/ketone by crystallization, distillation, extraction and/or chromatographic separation.
